(19) United States
(12) Patent Application Publication
(10) Pub. No.: US 2011/0118767 A1
(43) Pub. Date: May 19, 2011

(54) Method and Apparatus for Determining Status of Approximation Structures on Anastomosis Device

(75) Inventor: Kory P. Hamel, Bloomington, MN (US)

(73) Assignee: AMS RESEARCH CORPORATION, Minnetonka, MN (US)

(21) Appl. No.: 13/055,626

(22) PCT Filed: Jul. 30, 2009
(86) PCT No.: PCT/US09/52223

§ 371 Date: Jan. 24, 2011

Related U.S. Application Data
(60) Provisional application No. 61/084,698, filed on Jul. 30, 2008.

(51) Int. Cl. A61B 17/11

(52) U.S. Cl. 606/153

(57) ABSTRACT

An anastomosis device (10) in which radiographic band markers (26, 28) are used to determine fluoroscopically whether approximation structures (24) have been deployed based upon the observed alignment of the radiographic band markers and the approximation structures. The radiographic band markers are positioned proximate a distal end (22) of the anastomosis device wherein a medical professional can fluoroscopically compare a position of crimp hubs (30) on the approximation structures with the radiographic band markers.

### PRIORITY CLAIM

The present application claims priority to U.S. Provisional Application No. 61/084,698 filed Jul. 30, 2008 and entitled, “METHOD AND APPARATUS FOR DETERMINING STATUS OF APPROXIMATION STRUCTURES ON ANASTOMOSIS DEVICE,” which is hereby incorporated by reference in its entirety.

### FIELD OF THE INVENTION

The present invention relates generally to anastomosis devices and their associated methods of use in reconnecting tissue. More particularly, the present application relates to an improved anastomosis device to enhance overall patient safety during performance of anastomosis and other related surgical procedures including urethral procedures.

### BACKGROUND OF THE INVENTION

Anastomosis procedures are required for connecting or re-connecting certain body tissues, e.g., as part of a surgical procedure. Typically, these tissues define a body lumen such as a blood vessel, intestinal, digestive or urinary tissue that are severed and/or reconnected as part of a successful treatment. One representative example can include a radical prostatectomy procedure in which, a surgeon removes all or most of a patient's prostate. Because the urethra travels through the prostate immediately before reaching the bladder, the upper part of the urethra is also removed with the surgery. The procedure generally leaves a severed urethral stump and a severed bladder neck. To restore proper urinary functions, the bladder and the urethra must be reconnected.

Representative anastomosis devices and procedures describing the use of an anastomosis device in connecting a severed urethral stump and a severed bladder neck are described in U.S. Patent Publication Nos. 2004/0087995A1, 2005/0070938A1, 2005/0131431A1 and 2007/0219584A1, which are commonly assigned to the assignee of the present application, American Medical Systems of Minnetonka, Minn., and are incorporated by reference in their entirety. Through the use of a combination of retention features including an inflation balloon and a plurality of tissue approximating structures described as extendable tines, the urethral stump and bladder neck can be aligned and retained in contact throughout a healing period for the tissue. While the tissue of the urethral stump and bladder neck tissue are held together during healing, the anastomosis device also provides a drainage lumen allowing bodily fluids and other materials to pass during the healing period.

While the aforementioned anastomosis device and procedure effectively reconnects tissue during surgical procedures, it would be advantageous to improve upon the present designs and procedures to enhance the functionality, reliability and safety associated with use of anastomosis devices in medical treatment.

### SUMMARY OF THE INVENTION

The present invention comprises an anastomosis device which utilizes markers proximate a distal end of the anastomosis device such that a physician can fluoroscopically determine a deployment status of approximating structures on the anastomosis device. Generally, the distal end of the device includes impregnated radiographic bands in the device at the location of deployment hubs when the approximating structures are in a fully retracted position. Therefore, when a user is retracting the approximating structures from a deployed configuration, the user is able to quickly determine if the approximating structures have been completely retracted based on whether the hubs and radiographic bands are aligned.

In one aspect, the present invention is directed to an anastomosis device having markers proximate a distal end of the anastomosis device. One or more radiographic markers, which may take the form of bands, are positioned proximate the distal end such that a deployment state of the approximating structures, residing in a full or partial retracted state or full or partial extended state, can be fluoroscopically determined by a physician.

In another aspect, the present invention is directed to a method for verifying a deployment status of a retention structure on an anastomosis device. The method can comprise providing an anastomosis device having one or more radiographic markers at a distal end of the anastomosis device. The method can further comprise visualizing the distal end with a fluoroscopic device to compare a position of an approximating structure with the one or more radiographic markers. Anastomosis devices that include markers can advantageously eliminate difficulties in the anastomosis procedure by ensuring the approximated structure is in a retracted state prior to advancing, withdrawing, twisting or otherwise maneuvering of the anastomosis device. The ability to avoid maneuvering the anastomosis device with approximating structures in a deployed state provides significant advantages in patient safety during an anastomosis procedure.

Representative embodiments of anastomosis devices of the invention can include an elongate body, a tissue approximating structure, a drainage lumen, e.g., running as a channel within the elongate body and mechanisms for actuating, for example, deploying and retracting the tissue approximating structure. The tissue approximating structure and related actuating mechanisms are isolated from the drainage lumen. Radiographic markers disposed on the elongate body proximate a distal end of the elongate body allow the verification of a deployment state of the approximating structure with a fluoroscopic device.

In particular embodiments, radiographic markers can comprise a single band that aligns with an approximating structure, or a particular component of the approximating structure such as a hub that is not extended from the elongate body when in a deployed state. In other particular embodiments, the radiographic markers can comprise one or more parallel bands, which allow verification of a deployment status of the approximating structure and in particular, a retracted state when the approximating structure, or a particular component of the approximating structure, aligns between the parallel bands. In other particular embodiments, the radiographic markers can comprise alternative configurations such as, for example, one or more geometric shapes or repeating geometric shapes. In other particular embodiments, a mesh, surrounding a portion of the elongate body allows the deployment of the approximating structure while serving the dual purpose of acting as the radiographic marker.

According to the present description, the term “distal end” refers to a portion of an anastomosis device that is inserted into a body lumen during an anastomosis procedure such as tissue in the region of a bladder, urethra, urethral stump, or perineal wall. The term “proximate end” refers to a portion of an anastomosis device that is opposite from the distal end, including a portion that remains exterior to the body during use.

The terms “tissue approximating” and simply “approximating” refer to a process of binding, holding or otherwise placing body tissue in contact for healing. Examples include: the process of bringing severed surfaces of a bladder neck and a urethral stump, or two opposing severed urethral tissues, into contact for healing; and the process of holding severed surfaces of a bladder neck and a urethral stump, or two opposing severed urethral tissues, together for a period of time during which healing occurs.

The above summary of the invention is not intended to describe each illustrated embodiment or every implementation of the present invention. Rather, the embodiments are chosen and described so that others skilled in the art can appreciate and understand the principles and practices of the invention. The Figures and the detailed description that follow more particularly exemplify these embodiments.

### BRIEF DESCRIPTION OF THE DRAWINGS

These as well as other objects and advantages of this invention will be more completely understood and appreciated by referring to the following more detailed description of the presently preferred exemplary embodiments of the invention in connection with the accompanying drawings of which:

FIG. 1A is a perspective view of one embodiment of an anastomosis device of the prior art.

FIG. 1B is section view of the anastomosis device of FIG. 1A taken at line 1B-1B of FIG. 1A.

FIG. 2 is a plan view of a distal end of an anastomosis device according to an embodiment of the present invention.

FIG. 3 is a section view of the distal end of the anastomosis device of FIG. 2 with an approximation structure in a deployed configuration according to an embodiment of the present invention.

FIG. 4 is a section view of the distal end of the anastomosis device of FIG. 2 with the approximation structure in a retracted configuration according to an embodiment of the present invention.

FIG. 5 is a perspective view of the distal end of the anastomosis device of FIG. 2 according to an embodiment of the present invention.

FIG. 6 is a perspective view of a distal end of an anastomosis device including an integrated mesh in a connective sheath according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

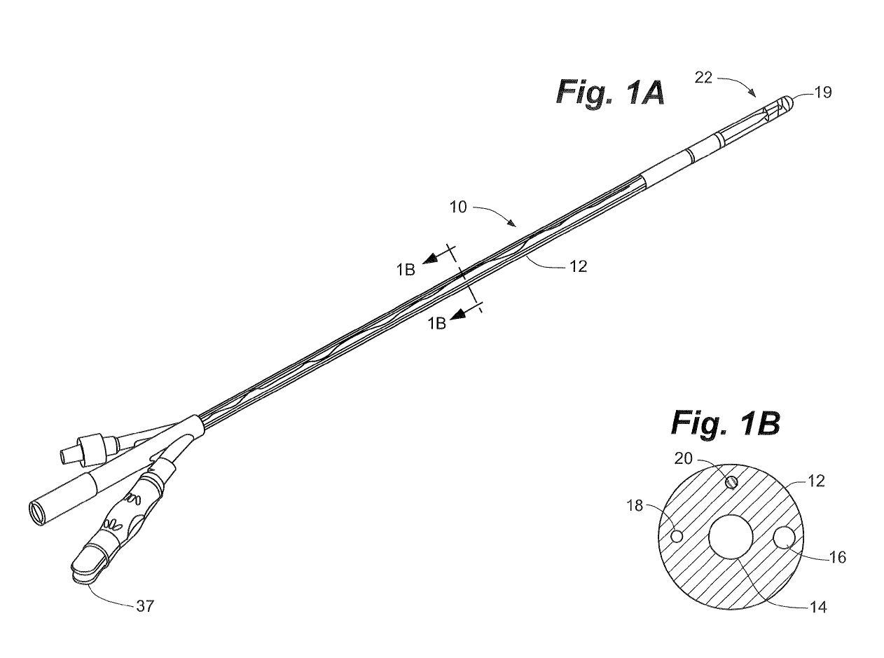

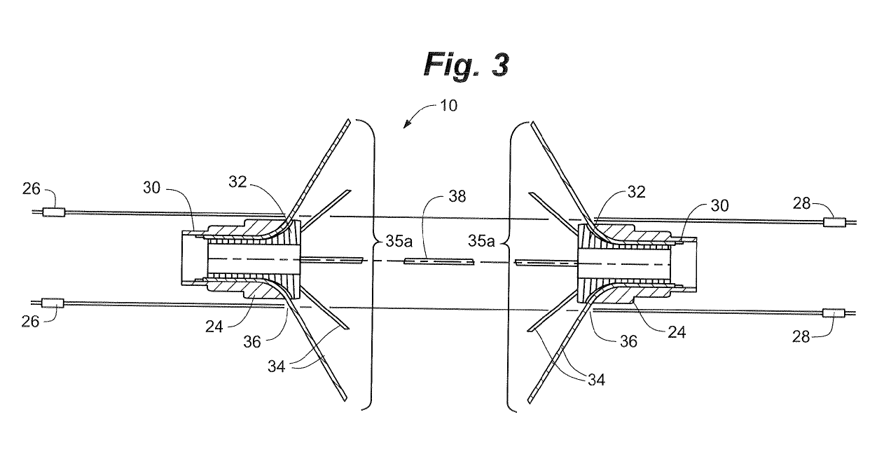

Throughout the several Figures, and referring initially to FIGS. 1A and 1B, exemplary embodiments of an anastomosis device 10 are illustrated, which comprise an elongated catheter shaft 12 having a central drainage lumen 14 that extends generally along the length of catheter shaft 12. Catheter shaft 12 further includes an actuation wire lumen 16 through which an actuation wire 38, shown in FIG. 3, for biasing tissue approximating structures can extend, and an inflation lumen 18 used for inflation and deflation of an inflation balloon near a distal tip 19 of the anastomosis device 10. Catheter shaft 12 can further include at least one cable or wire 20 that acts as a reinforcement structure and extends through the wall of catheter shaft 12 along at least part of its length. This cable or wire 20 is made of a material that provides added strength to the anastomosis device 10 to prevent or minimize stretching or deformation of catheter shaft 12 during manipulation of anastomosis device 10. In addition, wire 20 protects the integrity of the anastomosis device 10 if it is subjected to unanticipated loads, such as impact loads. Thus, cable or wire 20 is designed to be as thin and lightweight as possible so as to not add unnecessary weight or bulk to anastomosis device 10, while still providing the desired amount of protection for the anastomosis device 10.

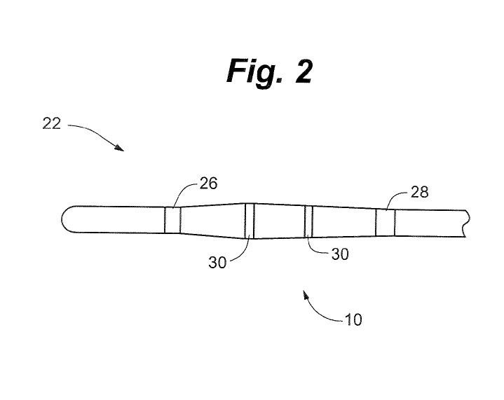

FIG. 2 discloses an anastomosis device 10 of the present invention including a distal end 22. As illustrated in FIG. 2, a pair of approximation structures 24 is in a completely retracted internal position and cannot be seen. A pair of marker bands 26 and 28 are found at positions aligned with the respective crimp hubs 30 of the approximation structures 24. Generally, the presence of marker bands 26, 28 assists physicians in determining the location and status of the approximation structures 24. This assessment of the deployment status of the approximation structures 24 is done fluoroscopically. The metal crimp hubs 30 can be viewed fluoroscopically and the marking bands 26 and 28 are radiographic bands and therefore are viewable as well. These marker bands 26 and 28 are impregnated into the anastomosis device 10 at the position of the crimp hubs 30 when the approximation structures 24 are in the fully retracted position. Therefore, when retracting the approximation structures 24 from a deployed state, a medical professional is able to determine if the tissue approximating structures 24 are in fact completely retracted by observing fluoroscopically whether the respective crimp hubs 30 and marker bands 26 and 28 are aligned.

Absent the ability to fluoroscopically observe the marker bands 26, 28, it can be very difficult for the medical professional to verify that the approximation structures 24 are completely retracted and that no portion of the approximation structures 24 remain partially deployed. Maneuvering the anastomosis device 10 such as, for example, advancing, withdrawing or twisting the anastomosis device 10 absent verification of this retracted state of the approximation structure 24 can cause difficulties in the anastomosis procedure.

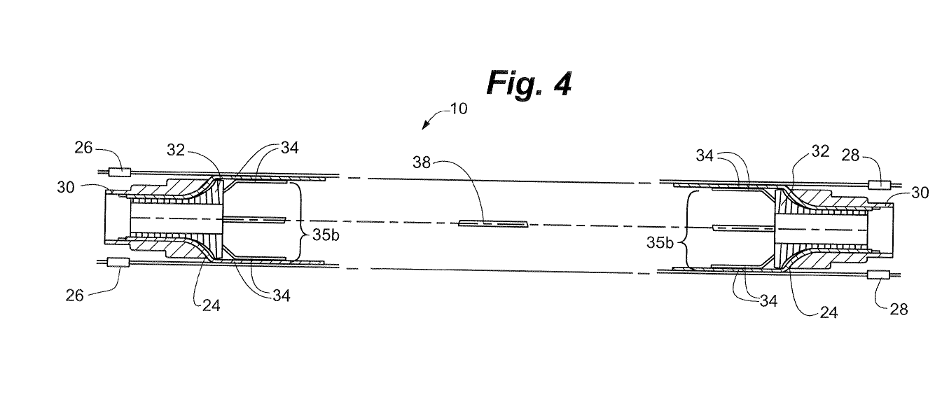

FIGS. 3 and 4 illustrate sectional views of the anastomosis device 10 and more specifically, depict operation of the approximation structures 24 and marker bands 26, 28 by a representative anastomosis device 10 of the present invention. Each tissue approximation structure 24 can be seen to include a crimp hub 30, deflectors 32 and tines 34. Apertures 36 are positioned proximate the distal end 22 of the catheter shaft 12. The crimp hubs 30 are generally located opposite the protruding tines 34. The tines 34 are used to contact and optionally penetrate into or through the one or more tissues of the bladder, bladder neck, urethra, bulbar urethra, urethral stump, or perineal floor, to place opposing severed tissue surfaces into contact for healing, and preferably also to retain the tissues in contact with each other during the healing period.

FIG. 3 sets forth a view where the tines 34 and approximation structures 24 are in a deployed configuration 35a. FIG. 4 sets forth a view where the tines 34 and approximation structure 24 are in a fully retracted configuration 35b.

To position the tissue approximating structures 24 in deployed configuration 35a, an actuation wire 38 manipulated at a biasing end 37 of the anastomosis device 10 (as seen in FIG. 1A) causes the tines 34 to extend forward and outward from the deflector 32 and the tines 34 are extended through apertures 36 in the distal body. The metal crimp hubs 30 of the approximation members 24 are accordingly drawn inward toward the deployed tines 34 and away from radiographic marker bands 26 and 28 impregnated into the anastomosis device 10, as seen in FIG. 3.

When the tissue approximating structures 24 are biased to retracted configuration 35b, the actuating wire 38 draws the tines 34 back towards the deflector 32. Accordingly, the crimp hubs 30 of the approximating structures 24 are moved into alignment with the radiographic marker bands 26 and 28 in the fully retracted configuration 35b, as seen in FIG. 4.

Through visualizing the relationship of crimp hubs 30 relative to the impregnated radiographic marker bands 26 and 28, a physician can determine whether or not the tines 34 have been fully retracted to the retracted configuration 35b such that the anastomosis device 10 can be advanced, removed or otherwise manipulated without danger to the patient.

The radiographic markers 26 and 28 may be a single band as shown in FIGS. 3 and 4, or alternatively, the radiographic markers 26 and 28 can comprise two or more parallel bands. The radiographic markers 26 and 28 can also be configured as geometric shapes, such as a square or circle that aligns with the entire tissue approximating structure, or a particular component of the tissue approximating structure, such as the crimp hub 30. Regardless of the configuration of the radiographic markers 26 and 28, the radiographic markers can be made of a continuous material or disconnected material (such as dots or lines), or a combination thereof.

While a preferred embodiment of the anastomosis device 10 involves the radiographic markers 26 and 28 being impregnated at a position relative to the crimp hubs 30 in the retracted position of the tissue approximating structure 24, the radiographic markers 26 and 28 can also be located at other positions relative to other components of the tissue approximating structure 24. Each tissue approximating structure 24 can also have more than one respective radiographic marker, such as a radiographic marker located at the position of the crimp hub 30 and another radiographic marker located proximate the apertures 36. Other positions and arrangements of radiographic markers are also envisioned.

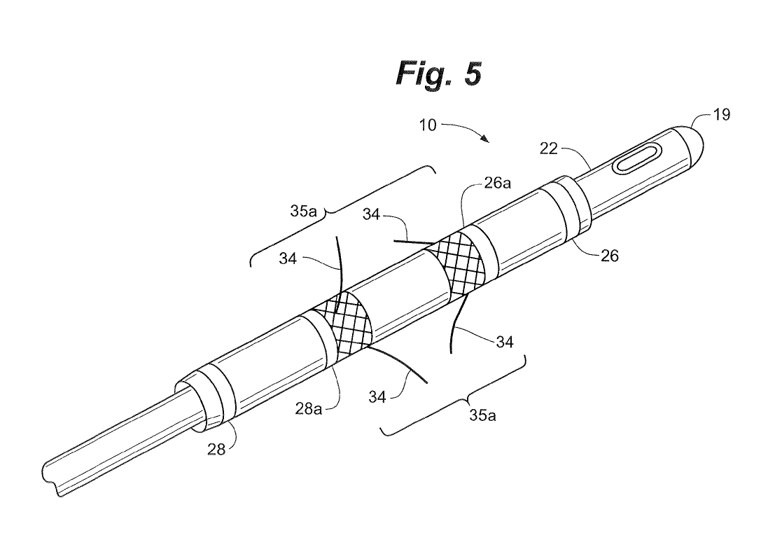

FIG. 5 illustrates anastomosis device 10 with tines 34 extended into the deployed configuration 35a. The relative positioning of the radiographic marker bands 26, 28 and tines 34 are illustrated for deployed configuration 35a along with the remaining components at the distal end 22 of the anastomosis device 10. Another representative embodiment of an anastomosis device 10 including more than one radiographic marker for each respective tissue approximating structure 24 is illustrated by including radiographic markers 26a and 28a in FIG. 5, which may be of the same or different material or configuration of radiographic marker bands 26 and 28.

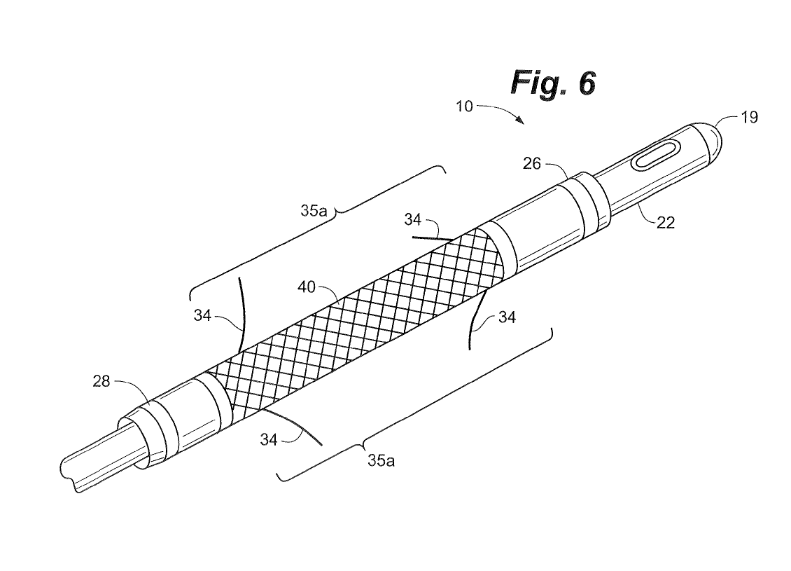

Another representative embodiment of an anastomosis device 10 including an integrated mesh feature 40 is shown in FIG. 6. Similarly to the prior embodiment, radiographic marker bands 26 and 28 are placed such that they will be in position for fluoroscopic viewing and determination of the deployed or non-deployed position of the tines 34 and approximation members 24. In this representative embodiment, mesh feature 40 can also be radiographic and serve a dual purpose such as to allow the deployment of the tines 34 into the deployed configuration 35a and also serve as a radiographic marker with or without other radiographic markers.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. An anastomosis device, comprising:
a catheter body having a proximal end and a distal end, wherein said catheter body includes an external surface, and internal surface and at least one approximating aperture;
at least one tissue approximating structure contained by the internal surface of said catheter body, wherein said tissue approximating structure includes a hub and an extendible and retractable tine; and
a radiographic marker positioned about the external surface of the catheter body, wherein alignment of the hub with the radiographic marker provides indication of the tine being in an extended or retracted position.

2. The anastomosis device of claim 1, wherein the tissue approximating structure is selectively extended or retracted from the approximating aperture with an actuating mechanism extending through an actuation lumen defined between the proximal end and the distal end of the catheter body.

3. The anastomosis device of claim 2, wherein the actuating mechanism selectively directs the hub such that the extendible and retractable tine is directed against a deflector located proximate the approximating aperture, wherein the extendible and retractable tine is directed out of the approximating aperture so as to assume and extended disposition.

4. The anastomosis device of claim 1, wherein the at least one tissue approximating structure comprises a distal-most approximating structure and a proximalmost approximating structure, and wherein the at least one approximating aperture comprises a distal-most approximating aperture and a proximal-most approximating aperture, and wherein an actuating mechanism selectively slidably directs hubs of both the distalmost and proximalmost approximating structures within the catheter body.

5. The anastomosis device of claim 4, wherein slidably approximating the distalmost and proximalmost approximating structures directs the corresponding extendible and retractable tines against the corresponding deflector such that the extendible and retractable tines are directed out of the corresponding distal-most and proximal-most approximating aperture such that the extendible and retractable tines assume an extended disposition.

6. The medical device probe of claim 5, wherein the distal-most approximating structure and the proximalmost approximating structure can be positioned independently inside the catheter body with the actuating mechanism.

7. The anastomosis device of claim 1, wherein each radiographic marker comprises at least one radiographic band surrounding the outer body wall.

8. The anastomosis device of claim 7, wherein each radiographic marker comprises two or more bands surrounding the outer body wall, wherein the two or more bands are arranged in parallel relation to each other.

9. The anastomosis device of claim 7, wherein the at least one radiographic band comprises a continuous band of radiographic material.

10. A method for verifying a retention status of an anastomosis device, comprising:
providing an anastomosis device having a catheter body with a proximal end and a distal end, the anastomosis device including at least one tissue approximating structure disposed within the catheter body and at least one radiographic marker positioned on an exterior wall of the catheter body; and
verifying a position of the at least one tissue approximating structures relative to the at least one radiographic marker.

11. The method of claim 10, wherein verifying that the at least one tissue approximating structure is aligned with the at least one radiographic marker indicates that retention tines on the at least one tissue approximating structure are withdrawn within the elongate flexible catheter body.

12. The method of claim 11, further comprising:
manipulating a position of the anastomosis device when the retention tines are withdrawn within the catheter body.

13. The method of claim 10, wherein verifying that the at least one tissue approximating structure is not aligned with the at least one radiographic marker indicates that retention tines on the at least one tissue approximating structure are at least partially extended from the catheter body.

14. The method of claim 13, further comprising:
avoiding positional manipulation of the anastomosis device when the retention tines are at least partially extended from the catheter body.

15. An anastomosis device, comprising:
a catheter body having a proximal end and a distal end, the distal end comprising an outer body wall and an inner body wall defining a hollow central channel, and an inner drainage lumen, each extending at least a portion of a length of the catheter body, the outer body wall including a drainage aperture at the distal end for communication with the proximal end through the inner drainage lumen, the outer wall further including a pair of approximating apertures;
an inflatable balloon positioned at the distal end of the catheter body,
a pair of tissue approximating structures disposed within the hollow central channel, each tissue approximating structure including a hub, a deflector and a set of protruding tines; and
a pair of spaced apart radiographic markers positioned on the outer body wall, wherein alignment of each radiographic marker with the corresponding hub of the corresponding tissue approximating structure is indicative of the protruding tines being in a fully retracted disposition within the inner body wall.
